(19) United States
(12) Patent Application Publication
(10) Pub. No.: US 2007/0095342 A1
(43) Pub. Date: May 3, 2007

(54) Accessorized inhaler

(76) Inventors: Bijan Olfati, Newport Beach, CA (US); Bahram Olfati, Richmond Hill (CA)

Correspondence Address: VOLPE AND KOENIG, P.C., UNITED PLAZA, SUITE 1600, 30 SOUTH 17TH STREET, PHILADELPHIA, PA, 19103, US

(21) Appl. No.: 11/252,777

(22) Filed: Oct. 18, 2005

(51) Int. Cl. A61M 11/00

(52) U.S. Cl. 128/200.23

(57) ABSTRACT

An apparatus for dispensing medicine has a housing for receiving the medicine and for covering the medicine. A dispenser extends from the housing for administering the medicine. The housing is cosmetically enhanced with at least one of a graphic and design indicia to convey a visual message beyond the immediate utility of the apparatus.

### FIELD OF INVENTION

This invention relates to medicine dispensing apparatus. More particularly this invention relates to oral mist applicators.

### BACKGROUND

Asthma is a fact of life for many children and adults. As many as one in five Canadian children have asthma. In the U.S., 20.3 million Americans reported having asthma in 2001. This represents an increase of more than 60% since the early 1980's. The largest proportion of sufferers are children. This demographic is reflected in other countries. Findings show that the number of asthma suffers worldwide is rising within the western hemisphere countries to the extent that, at a conservative estimate, potentially 60 million people in the western hemisphere suffer asthma.

Asthma has a number of treatments requiring a number of different methods of delivery/applicators. The most common delivery method is an oral mist applicator whereby the medicine is delivered to an asthma sufferer's lungs via a gas-powered mist diffuser. Many sufferers, in particular children, need more than one type of medicine and accordingly may require more than one applicator (also referred to as an “inhaler” but the former term is used herein).

At present, the principal suppliers of applicators are the same companies that manufacture the various asthma drugs. The applicators are generally packaged with the drug. Accordingly, given that the asthma sufferer is buying the drug rather than the applicator, the applicator tends to be a relatively utilitarian appliance and quite clearly has the look of a “medical device”.

Frequently, asthma sufferers have need for their applicators when in public. In view of the utilitarian, medical appliance appearance of conventional inhalers, this causes many inhaler users to feel uncomfortable and embarrassed. Given the health and fitness goals of the general population which strive toward “perfection” of one's body, taking medication in public makes many people feel uncomfortable. Worse yet is that the method of using an inhaler is such an obvious way of taking medication, as inhalers generally have to be lifted to the front of one's face with the recipient's torso upright.

It is an object of the present invention to provide an asthma inhaler/applicator (herein referred to as “applicator”) which would be more acceptable to users by avoiding at least some of the discomfort associated with the appearance and configuration of current designs.

### SUMMARY

In general terms, the present invention seeks to accessorize apparatus for dispensing medicine, such as asthma applicators, through the use of cosmetic enhancements to the color, finish, branding (other than the drug name branding) and/or physical design of the applicator to achieve the result of the applicator's appearance becoming less overtly medical or “non-medical”.

In accordance with the present invention, an apparatus is provided which has a housing for receiving the medicine and covering the medicine, with a dispenser extending from the housing for administering the medicine. The apparatus further has at least one of graphic and design indicia for cosmetically enhancing the housing to convey a visual message beyond the immediate utility of the apparatus.

The housing may be an asthma applicator which receives a canister of asthma medicine and the dispenser may be a mouthpiece fluidly communicating with the canister when the canister is housed in the apparatus.

The graphic and design indicia may include a time of day (day/night) indicator; designer branding; a metal-like finish; ornamental graphic elements; monograms; sports related branding and entertainment related branding.

The housing may have a shape emulating a non-medically related structure.

The non-medically related structure may include a cosmetic container, a perfume container, a cartoon character, a consumer product container not associated with medicines, miniature sports equipment articles and virtually any other article capable of being configured so as to dispense medicine and having a shape which masked the principal utilitarian function of the apparatus.

### BRIEF DESCRIPTION OF THE DRAWING(S)

The invention will be described below with reference to the accompanying illustrations in which:

FIGS. 1A-1D are different views of a conventional “prior art” drug company supplied inhaler;

FIG. 2A is a perspective view of an alternate prior art inhaler design;

FIG. 2B is an exploded view of the applicator of FIG. 2A;

FIG. 3A is a perspective view of housing configuration for an asthma applicator;

FIG. 3B is an exploded view of the applicator of FIG. 3A;

FIGS. 4 through 11 are front elevational views illustrating a sampling of graphic and design indicia applied to an asthma applicator housing in accordance with the present invention.

### DETAILED DESCRIPTION OF THE PREFERRED EMBODIMENT(S)

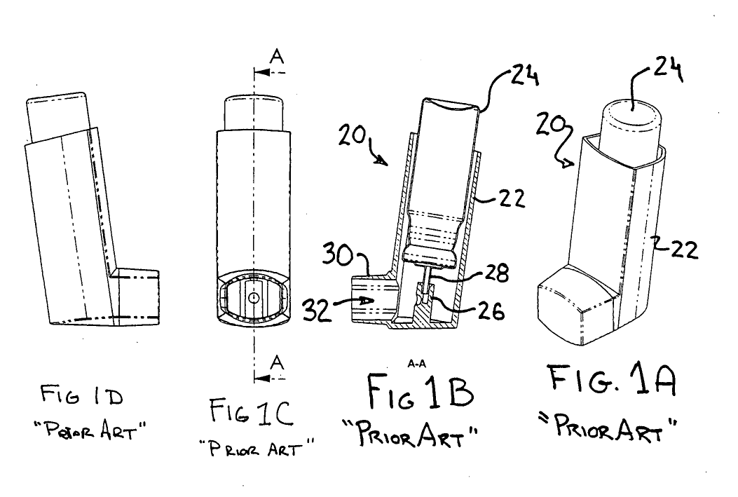

An apparatus for dispensing medicine, more particularly an asthma applicator of convention configuration is generally illustrated 20 in FIGS. 1A-1D. The apparatus 20 has a housing 22 which receives a medicine canister 24. The housing has a receptacle 26 for receiving a nozzle 28 extending from the canister 24 through which medicine is dispensed from the canister into the inhaler.

Although the illustrations show asthma applicators having gas based delivery systems, it will be appreciated that more and more asthma applicators are being introduced to the market in different shapes and different delivery methods. For example, the applicator may powder a pill or aspirate a powder rather than using a gas propellant. Also, some applicators have different overall shapes than the ones illustrated. The present invention is limited to gas based delivery systems or any particular configuration and the disclosure and claims should be read in that context.

A mouthpiece 30 extends from the housing 22 and has a passage 32 which fluidly communicates through an aperture 34 with the nozzle 28. In use, the medicine canister 24 is pressed toward receptacle 26 releasing a valve (not illustrated) causing pressurized gas to dispense medicine through the aperture 34 into the passage 32. A user places their mouth over the mouthpiece 30 and inhales a medicine into their lungs.

As can be seen from FIGS. 1A and 1B, the prior art device makes no attempt to mask that it is a medical device for dispensing asthma medication. In fact, the medicine canister 24 is visible through the top of the device.

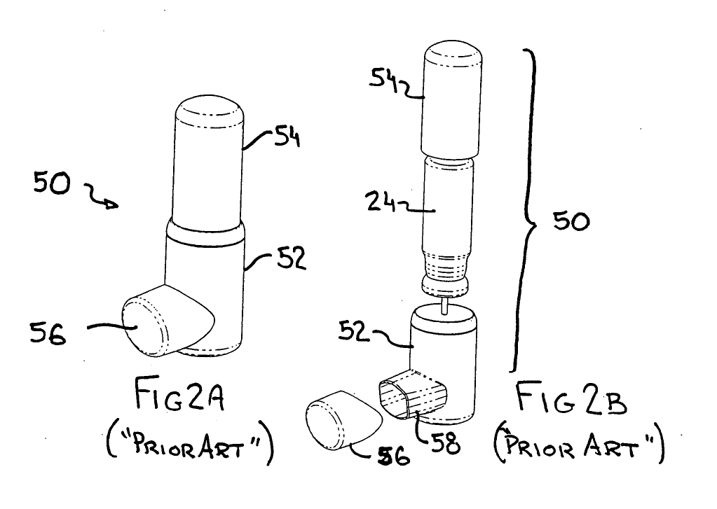

More recently, I have designed a fully contained housing which covers the entire medicine canister 24 so as to at least avoid the look of an apparatus which is little more than a spray nozzle for a medicine canister 24. A first embodiment of my design is generally indicated by reference 50 in FIGS. 2A and 2B. The internal workings of the apparatus 50 are generally the same as described above with respect to the FIGS. 1A and 1B embodiment. As can be seen from the exploded view of FIG. 2B, the fully enclosing housing 50 includes a base member 52 analogous to housing 22 of the earlier design. In addition however, the apparatus 50 includes a top member 54 which covers a base of the medicine canister 24 and a cap 56 for covering a mouthpiece 58. Although the FIGS. 2A and 2B design is clearly an asthma applicator, it includes features, such as the top member 54, which at least take the medicine canister 24 and hide it out of sight.

A more recent design, along the lines of the FIGS. 2A and 2B design is illustrated in FIGS. 3A and 3B. The FIGS. 3A and 3B design seeks to further avoid the appearance of a conventional asthma applicator by deviating from the more conventional generally cylindrical shape. Referring to FIGS. 3A and 3B, the applicator is generally indicated by reference number 70. The applicator has a base member 72 for receiving the medicine canister 24, a top member 74 for hiding the base of the medicine canister 24 and a cap 76 for covering a mouthpiece 78 which extends from the base member 72 and fluidly communicates with the medicine canister 24.

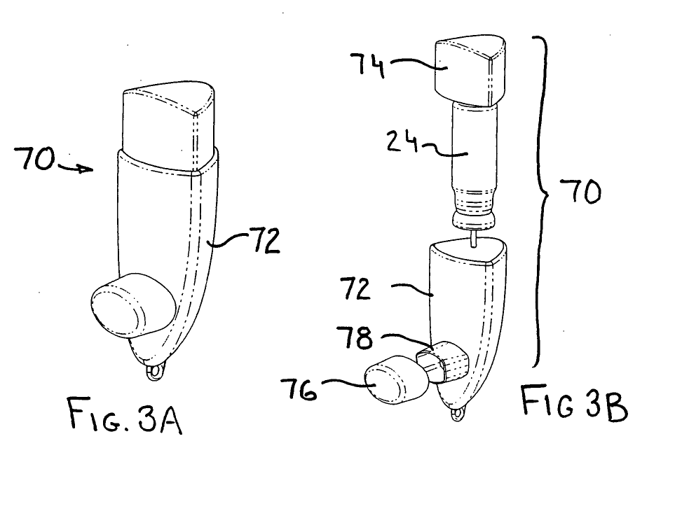

FIGS. 4 through 11 illustrate a radical departure from current applicator design philosophy. Unlike prior designs which either communicate nothing or seek to hide the medicine canister 24, the designs illustrated convey a bold statement through the use of graphic and design indicia. FIGS. 4 through 11 are merely samples of what may be accomplished by applying applicant's invention which in effect removes an asthma applicator from the realm of a medical appliance to that of a designer accessory. The various effects are discussed individually below.

FIG. 4 and FIG. 5 disclose respective children's day and night designs. The applicator of FIG. 4 is generally indicated by reference 80 and has a sun 82 and clouds 84 against a sky blue applicator. In contrast, the applicator of FIG. 5, which is generally indicated by reference 90 has a black background 92 depicting a night sky with stars 94. The logic of the day applicator 80 and the night applicator 90 is for use in situations where children require different asthma medication for day time and night time use.

FIG. 7 illustrates an applicator 100 which borrows from cosmetic design container indicia. In particular, the container utilizes a design of the type associated with Chanel (trademark) perfumes and has a black lacquered finish 102 set off by gold accent bands 104.

FIG. 6 illustrates an applicator 110 having a “chrome effect finish” 122. The applicator 110 may be of metal or injection moulded plastic with chrome plating applied to its surface to achieve the chrome effect.

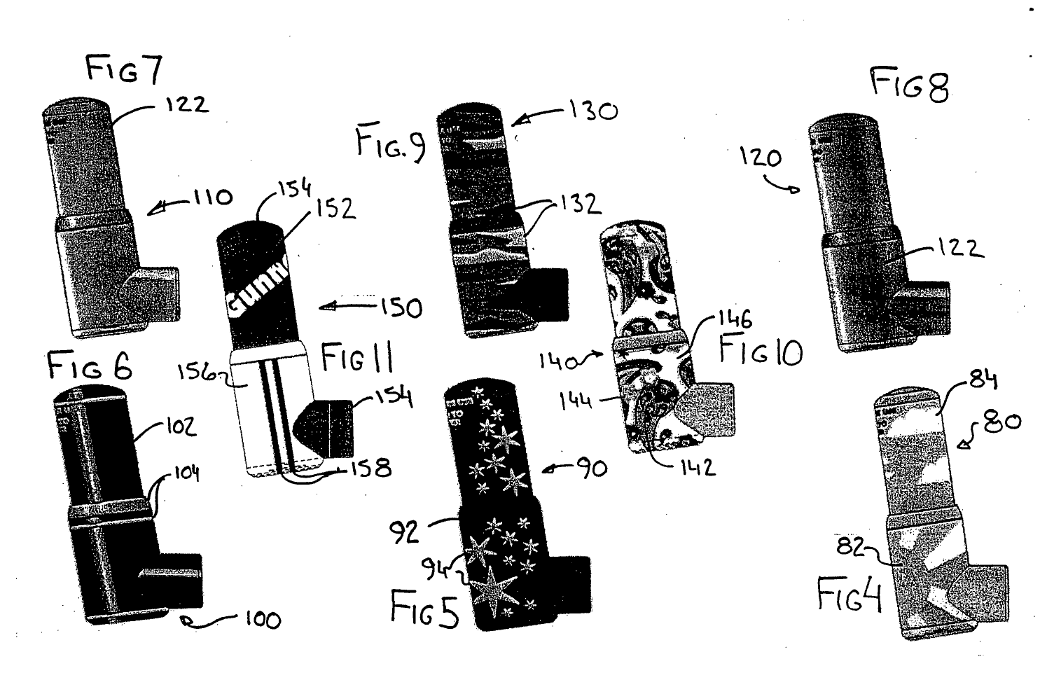

FIG. 8 discloses an applicator 120 similar to that of FIG. 9 however the finish 122 may be a bare metal such as brushed stainless steel, copper or paint emulating the foregoing effects.

FIG. 9 illustrates an applicator 130 having camouflage striping 132 to achieve a “camouflage finish”. It is expected that the camouflage finish would be popular with teenagers.

FIG. 10 illustrates an applicator 140 having a design which may appeal to girls. The design includes paisleys 142 and flowers 144 against a neutral background 146.

FIG. 11 illustrates an applicator 150 having a sport effect finish. The effect may be achieved by having a logo 152 printed on the applicator 150. Additionally, an athletic team's colors may be used such as having a first color 154 for the top member and mouthpiece and a second color 156 applied to the base member. Additionally, stripes 158 of the first color 154 may be applied to the base member.

Other variants are possible. One example would be team logos or indicia relating to the entertainment industry. For example, cartoon characters might be depicted, or possibly even the entire shape of the housing might emulate a cartoon character.

The above is intended in an illustrative rather than a restrictive sense. Many other design variations are possible while staying within the spirit and scope of the present invention as defined by the claims set out below. Insofar as such variants are within the wording of the claims set out below, they are to be considered as being within the scope of this patent.

### PARTS LIST

20 conventional apparatus
22 housing
24 medicine canister
26 receptacle
28 nozzle of canister
30 mouthpiece
32 passage (mouthpiece)
34 aperture
50 1 fully enclosing design
52 base member
54 top member
56 cap
58 mouthpiece
70 2 fully enclosing design
72 base member
76 cap
78 mouthpiece
80 FIG. 4 applicator
82 sun
84 clouds
86 blue background
90 FIG. 5 applicator
92 black background
94 stars
100 FIG. 6 applicator
102 black laquer finish
104 gold accent bands
110 FIG. 7 applicator
112 chrome effect
120 FIG. 8 applicator
122 metal
130 FIG. 9 applicator
132 camouflage striping
140 FIG. 10 applicator
142 paisleys
144 flowers
146 neutral background
150 FIG. 11 applicator
152 logo
154 1 color
156 2 color
158 stripes

What is claimed is:

1. An apparatus for dispensing medicine comprising:
a housing for receiving said medicine, said housing covering said medicine;
a dispenser extending from said housing for administering said medicine; and,
at least one of graphic and design indicia for cosmetically enhancing said housing to convey a visual message beyond the immediate utility of said apparatus.

2. The apparatus of claim 1 wherein:
said housing receives a canister of asthma medicine; and,
said dispenser is a mouthpiece fluidly communicating with said canister when said canister is housed in said apparatus.

3. The apparatus of claim 2 wherein said at least one of said graphic and design indicia is a member selected from the group consisting of: a time of day indicator; designer branding; a metal-like finish; ornamental graphic elements; monograms; sports related branding and entertainment related branding.

4. The apparatus of clam 2 wherein said housing has a shape emulating a non-medically related structure.

5. The apparatus of claim 4 wherein said structure is a member selected from the group consisting of: a cosmetic container; a perfume container; a cartoon character; a consumer product container not associated with medicines; miniature sports equipment articles.
